(19) United States
(12) Patent Application Publication
(10) Pub. No.: US 2015/0173468 A1
(43) Pub. Date: Jun. 25, 2015

(54) IMPLANTABLE AND MAGNETIC RETENTION SYSTEM FOR REMOVABLE ATTACHMENT OF JEWELRY, ORNAMENTS AND OTHER WEARABLE FIXTURES

(71) Applicant: Wendy L. Stevenson, Canyon Country, CA (US)

(72) Inventor: Wendy L. Stevenson, Canyon Country, CA (US)

(21) Appl. No.: 14/578,886

(22) Filed: Dec. 22, 2014

Related U.S. Application Data
(60) Provisional application No. 61/920,095, filed on Dec. 23, 2013.

(51) Int. Cl. A44C 15/00; A61M 37/00

(52) U.S. Cl. CPC A44C 15/0005; A61M 37/0069

(57) ABSTRACT

An implantable magnetic retention system includes an implantable portion configured to be implanted subdermally or subcutaneously within a patient. The implantable portion includes a magnetic or ferromagnetic inner portion and a biocompatible outer portion fully enclosing the inner portion. A non-implantable portion is configured to be attached to a wearable ornament. The non-implantable portion includes a magnetic or ferromagnetic portion. At least one of either the implantable portion or the non-implantable portion includes the magnetic portion. Therefore, the implantable portion and the non-implantable portion are magnetically attracted to one another.

### CROSS-REFERENCE TO RELATED APPLICATIONS

This application claims priority to provisional application 61/920,095 filed on Dec. 23, 2014, the contents of which are fully incorporated herein with this reference.

### DESCRIPTION

1. Field of the Invention

The present invention generally relates to jewelry. More particularly, the present invention relates to an implantable and magnetic retention system for removable attachment of jewelry.

2. Background of the Invention

It is well known that various body pierced barbell-style studs have been worn for years as methods of decoration and self-expression. This includes all types of jewelry, earrings, tongue piercings and the like. Typically, these involve an outpatient procedure involving creating a hole, which eventually heals wherein a temporary stud can be removed and then an article or earring, such as an ear piercing-type earring, can be worn. Such body piercing and methods of decoration have exploded in recent years to cover nose piercings, tongue piercings, nipple piercings and the like.

There are some significant disadvantages to such piercings. One example would be a nose piercing or a diamond or other type of jewelry as worn on the outside of the nose. A young person may enjoy this for a few years and then decide that this is not such a good idea in a professional environment. The problem is once the piece of jewelry is removed, one is now left with a completely cured hole right through their nose. Similar analogies exists for the tongue, the nipples or any other part of the body where a piercing type of jewelry is used.

Another problem with all types of piercing with articles of jewelry or ornamentation, has to do with if the item gets snagged or ripped out from accidents. This can literally rip the earlobe, a nipple, the tongue or any other body part to which the piercing ornament has been affixed. There are also significant infection issues with piercings, particularly piercings around the tongue. In fact, any piercings can become infected if it is not properly cared for.

There is another category of jewelry and personal decoration that involves bracelets worn around the neck, the wrist or the ankles. Generally, these types of jewelry involve a clasp-type of arrangement, where the pendent, diamond or the like that would hang down on it, for example, below the neck. There is a disadvantage to all these forms of jewelry, in that the clasp arrangement (no matter what type of clasp is used) has a different mass and weight than the very thin and delicate light weight gold chain or the like. What happens, as the piece of jewelry is worn, the light weight chain tends to move around, so now you have the clasp either half way or all the way down the neck or adjacent to the piece of ornamental jewelry. What is needed is a methodology of keeping the clasp in its proper place.

The present invention solves all of the problems by providing a method of attaching jewelry or other decorations without the use of piercings. The present invention also solves the problem of keeping delicate chains and necklaces oriented in their proper position without a corresponding clasp ending up in the frontal visual field. The present invention also allows the placement of jewelry or decorations literally anywhere one can imagine without the need of a necklace, chain or piercing. The present invention also solves the problem if there is an inadvertent accident, wherein the item of jewelry or decoration is snagged, hit or ripped off. The present invention will allow for a release force, such that there will be no damage to underlying tissues.

The present invention resides in biocompatible implantable magnets, and/or magnetizable (ferromagnetic) materials that can be inserted as a thin wafer or structure into literally any part of the body subcutaneously or subdermally. A corresponding magnet or magnetizable material then becomes a part of the jewelry or items of decoration, such that, one simply sticks it over the magnet where it self-affixes onto the surface of the body on the exterior of the skin.

### SUMMARY OF THE INVENTION

An implantable magnetic retention system includes an implantable portion configured to be implanted subdermally or subcutaneously within a patient. The implantable portion includes a magnetic or ferromagnetic inner portion and a biocompatible outer portion fully enclosing the inner portion. A non-implantable portion is configured to be attached to a wearable ornament. The non-implantable portion includes a magnetic or ferromagnetic portion. At least one of either the implantable portion or the non-implantable portion includes the magnetic portion. Therefore, the implantable portion and the non-implantable portion are magnetically attracted to one another.

The implantable portion may be separate and distinct from the non-implantable portion, meaning the two portions are not physically connected and may be manufactured separately.

The implantable portion and the non-implantable portion may exert between 0.5 to 4.5 lbs of pull force when abuttingly disposed to one another.

The wearable ornament may include a necklace, a finger ring, a toe ring, an eyebrow ring, a belly ring, a nipple ring, a precious gemstone, an ornamental figure, a trinket or a pair of eyeglasses.

The biocompatible outer portion may be biocompatible, biostable and non-toxic. The biocompatible outer portion may include a first layer of copper, where the copper is fully enclosed by a second layer of nickel, where the nickel fully enclosed by a third layer of gold. The gold layer may be at least 50 millionths of an inch thick throughout.

The implantable portion may have at least one hole disposed therethrough or may have a plurality of holes disposed therethrough. The implantable portion may have at least one rib formed therein, wherein the rib is straight, curved, circular or spiral shaped. The implantable portion may be ring shaped. The implantable portion may include a microporous material.

The implantable portion's biocompatible outer portion may be a top portion sealed to a bottom portion by a seal. The seal may be a laser weld, a glass seal or a precious metal braze. The top portion and bottom portion may both be titanium, gold or ceramic. At least one of the top or bottoms portions may have a surface area two times, four times or ten times the surface area of the inner portion.

The implantable portion may further have a biocompatible mesh substrate attached to the outer portion.

The biocompatible outer portion may include a first layer fully enclosed by a second layer, where the first layer comprises a biomedical sputter or deposited coating. The first layer may be alumina ceramic. The second layer may be a plasma etched vapor deposite paralyne, a titanium, a silicone polymer, a non-toxic epoxy, a medical grade polyurethane, or a U.V. curable medical acrylic copolymer.

The biocompatible outer portion may include an anticoagulant, an antibiotic or a tissue in-growth promoters.

### BRIEF DESCRIPTION OF THE DRAWINGS

The accompanying drawings illustrate the invention. In such drawings:

FIG. 1 illustrates a prior art front view of a woman wearing a delicate necklace around her neck;

FIG. 2 illustrates a close-up view of a necklace and a typical clasp as previously illustrated in FIG. 1 now with the present invention;

FIG. 3 is similar to FIG. 2 now illustrating another embodiment of the present invention;

FIG. 4 is a rear view of a women having the present invention implanted into the back of her neck;

FIG. 5 is a rear view similar to FIG. 4 now showings the necklace being held in place by the present invention;

FIG. 5A is an enlarged view of the structure taken from lines 5A-5A from FIG. 5.

FIG. 6 illustrates a novel implant of the present invention;

FIG. 7 illustrates another novel implant of the present invention;

FIG. 7A illustrates another novel implant of the present invention;

FIG. 7B illustrates another novel implant of the present invention;

FIG. 8 illustrates a sectional view of any of the implants of the present invention;

FIG. 9 illustrates another novel implant of the present invention now showing a toroid shape;

FIG. 10 is a sectional view of the structure of FIG. 9 taken along lines 10-10;

FIG. 11 illustrates another novel implant of the present invention;

FIG. 12 illustrates another novel implant of the present invention;

FIG. 13 is a sectional view of the structure of FIG. 12 taken along lines 13-13;

FIG. 14 illustrates another novel implant of the present invention;

FIG. 15 illustrates another novel implant of the present invention;

FIG. 16 illustrates a perspective view of the human ear with the implant of the present invention implanted subdermally or subcutaneously within the earlobe;

FIG. 16A is similar to FIG. 16 now showing the jewelry attached;

FIG. 16B is a sectional view of the structure of FIG. 16A through lines 16B-16B;

FIG. 17 illustrates a perspective view of the human nose with the implant of the present invention implanted subdermally or subcutaneously within the outer nostril;

FIG. 17A is similar to FIG. 17 now showing the jewelry attached;

FIG. 17B is a sectional view of the structure of FIG. 17A through lines 17B-17B;

FIG. 18 is a front view of a woman's face with various implants of the present invention with its associated jewelry;

FIG. 19 is a front view of a woman's lower torso with various implants of the present invention with its associated jewelry;

FIG. 20 is a front view of a woman's upper torso with various implants of the present invention with its associated jewelry;

FIG. 21 is another novel embodiment of the present invention coupled to a woman's nipple or breast area;

FIG. 22 is a side view of a person with various implants of the present invention around his ear; and

FIG. 23 is a view similar to FIG. 22 now showing a pair of glasses being attracted to the implant of the present invention.

### DETAILED DESCRIPTION OF THE PREFERRED EMBODIMENTS

Magnetic implants have been used for several years in dentistry and reconstructive surgery, but their inclusion in the body modification world has been quite a recent one. Having magnets implanted under the skin allows the wearer to attach magnetic items to the outside of the skin. Some in the past have implanted magnets, but they weren't very strong and were only capable of picking up small items. Many who first attempted to have magnetic implants have gone on to develop problems with the implants which have necessitated their removal. The main problems have been caused by the rupturing of the silicone covering, bringing the metal into contact with bodily tissues, which in most cases caused it to rapidly break down. Attempts then were to use injection-molded silicone rather than the dip-coating used in the first generation. This molding provided an even coat over the pill-shaped magnet, rather than bubble shape of the dip-coated magnet, which lead to thin spots where the silicone was more likely to break down and cause failure. However, this is still not an ideal design as rejections are still occurring where the body attempts to push out the implant. Rejections are the regular risk of any implant or piercing, as the body is naturally inclined to push them out if they do not encapsulate with scar tissue.

Magnetic materials are well known in the art. Many of these materials are relatively inexpensive iron based alloys that can be permanently magnetized and then utilized as magnets to provide attraction or repellant magnetic forces in a wide variety of articles and devices. Other alloys are also known. One particular conventional alloy known as Alnico contains iron (Fe), nickel (Ni), aluminum (Al) and cobalt (Co), while another, known as Vicalloy, includes Fe, Co and Vanadium (V). One typical use of magnets is disclosed in U.S. Pat. No. 4,893,980 wherein inner and outer samarium (Sm)—Co magnets are used to impart sliding movement to a component of the device, while another use of such magnets is disclosed in U.S. Pat. No. 4,451,811. Examples of known permanent magnet materials include alloys of Neodymium-Iron-Boron (NdFeB), alloys of Aluminum-Nickel-Cobalt (AlNiCo), and Samarium Cobalt (SmCo). Bonded permanent magnet may be flexible or rigid, and consist of powdered NdFeB, Ferrite, or SmCo permanent magnet materials bonded in a flexible or rigid substrate of e.g., rubber nitrile, polyethylene, epoxy, polyvinyl chloride, silicone, rubber, or nylon. The forming of the magnet may be achieved by extrusion, compression molding, injection molding, calendering or printing. Bonded magnets enable unique flexible designs, and durable high tolerance shapes that are otherwise difficult to achieve.

Most of the current applications for magnets in jewelry items are generally for simple attachment of two components so that the item can be attached to clothing or ear piercing. For example, U.S. Pat. No. Re-35,511 discloses the use of common magnets to join two separate portions of an earring together (see FIG. 10), while U.S. Pat. Nos. 6,282,760 and 5,921,110 disclose using complementary magnets for attaching jewelry items to each other and to support devices, particularly for attachment of the items to clothing.

Traditionally, fine jewelry pieces are made of valuable precious metals or alloy materials thereof. These materials are based on gold, silver, palladium, platinum, rhodium, and lustrous alloys of these materials. Certain alloys may be heat-treated to increase strength or hardness, but generally these alloys are not magnetized. Certain of these alloys have no magnetic properties at all while the magnetic properties of others have not be utilized in fine jewelry pieces.

Magnetic alloys are very atomically structured and are inherently brittle. When magnetic alloys are thin, they are fragile. Small, thin components for jewelry made from magnets, including known precious metal magnets are too brittle for everyday use for jewelry. In fact, for jewelry made from magnets, including known precious metal magnets are too brittle for everyday use for jewelry. In fact, for jewelry, only thick magnetic parts have been inlayed or set in place in jewelry to utilize the forces from their magnetic fields. Consequently, magnetic alloys have very limited, non-aesthetic uses in jewelry applications. And while there have been precious metal magnetic materials, they have not been applied to fine jewelry.

U.S. Pat. No. 4,853,048 discloses that a known precious metal magnet of platinum-cobalt (Pt—Co) has equal atomic amounts of Pt and Co (representing about 77 weight percent Pt and 23 weight percent Co), but rejected its use stating that it has little value” in jewelry because its Pt content is below 85 weight percent. To make a jewelry component, their resolution of the problem was to add gold (Au) to form a ternary Au—Pt—Co alloy that contains 50 to 75% Co. Also, small amounts of Fe, Ni, copper (Cu), palladium (Pd), and silver (Ag) can be added to modify the properties of the ternary alloy. It was suggested that the resultant alloy material could be formed into a chain that can be magnetized in the direction of its thickness.

The magnetic properties of other alloys that contain precious metals have been investigated in a number of patients. U.S. Pat. No. 4,221,615 discloses soft-magnetic (i.e., non-permanent magnet) Pt—Co alloy products. U.S. Pat. No. 3,860,458 discloses a magnetic material consisting essentially of 40 to 60 atomic percent Pt, 45 to 55 atomic percent Co, and between 4 and 15 atomic percent Pt, 45 to 55 atomic percent Co, and between 4 and 15 atomic percent iron alone or with up to 5 atomic percent Ni, and optionally with up to 5 atomic percent Cu. U.S. Pat. No. 4,983,230 discloses magnetic alloys formed from Pt, Co, and Boron (B). U.S. Pat. No. 3,591,373 discloses a permanent magnetic alloy comprising 15-40 atomic percent Pt, 5-35 atomic percent Au and 40 atomic percent Fe. U.S. Pat. No. 3,755,796 discloses Co alloys that contain one of arsenic (As), germanium (Ge), indium (In), osmium (Os), Pt, rhodium (Ro), rhenium (Rh), ruthenium (Ru), silicon (Si), or Ag. U.S. Pat. No. 4,444,012 discloses Pt—Fe alloys that contain Co, Ni, H, Au, Ag, Cu, Iridium (Ir), Os, Pd, or Rh can be heat treated to provide magnetic properties, while U.S. Pat. No. 4,396,441 discloses permanent magnets of Pt—Fe alloys. U.S. Pat. No. 4,650,290 discloses a magneto-optical layer of a Pt-manganese-antimony alloy. U.S. Pat. No. 3,961,946 discloses magnetic Pt—Ni and Pt—Ni—Co alloys. U.S. Pat. No. 4,536,233 discloses permanent magnets of Sm—Co—Cu—Fe that also contain zirconium, titanium, hafnium, tantalum, niobium, and vanadium. Finally, U.S. Pat. No. 6,171,410 discloses hard (or permanent) magnetic alloys of patents, however, none of the properties or usefulness of these alloys for jewelry applications was investigated or discussed. Also, while British patent GB-1,067,054 discloses various heat treatments for Pt—Co alloys, it does not disclose any uses of such heat-treated materials in jewelry applications. Other examples, of known permanent magnet materials include alloys of neodymium-iron boron.

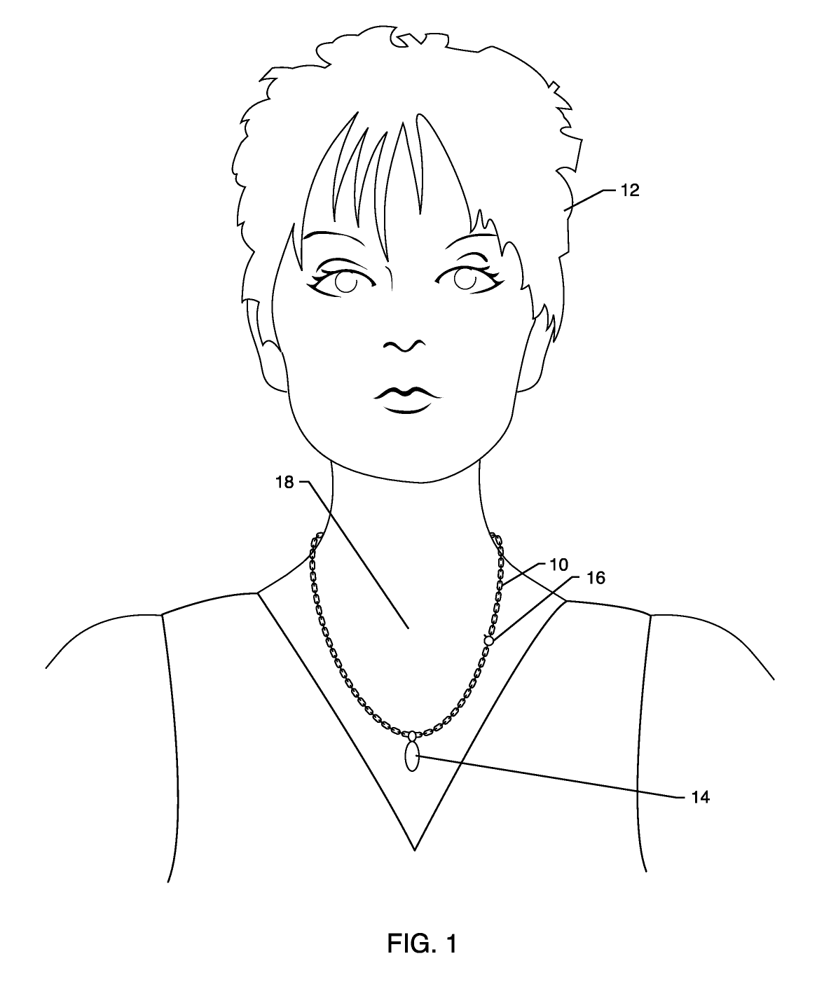

FIG. 1 is a prior art front view of a woman 12 wearing a delicate necklace 10 around her neck 18. There is an optional pendant 14, which can be an item of jewelry, an ornament or even an identification badge. The necklace 10 has a metal clasp structure 16 as shown. A problem in the prior art is that the mass of the clasp was always greater or different than the mass along the length of chain 10. Throughout the day, as the woman moves, the clasp has a way of working its way around from the back of the neck, which is its ideal location, around to the side or even all the way to the bottom adjacent to pendant 14. All one has to do is observe any woman in a crowded place and you will see the constant adjustments she is making to the necklace around her neck to keep putting the clasp back in place. Ideally, the clasp 16 should be located behind the neck (not shown) vertically in line with the spine.

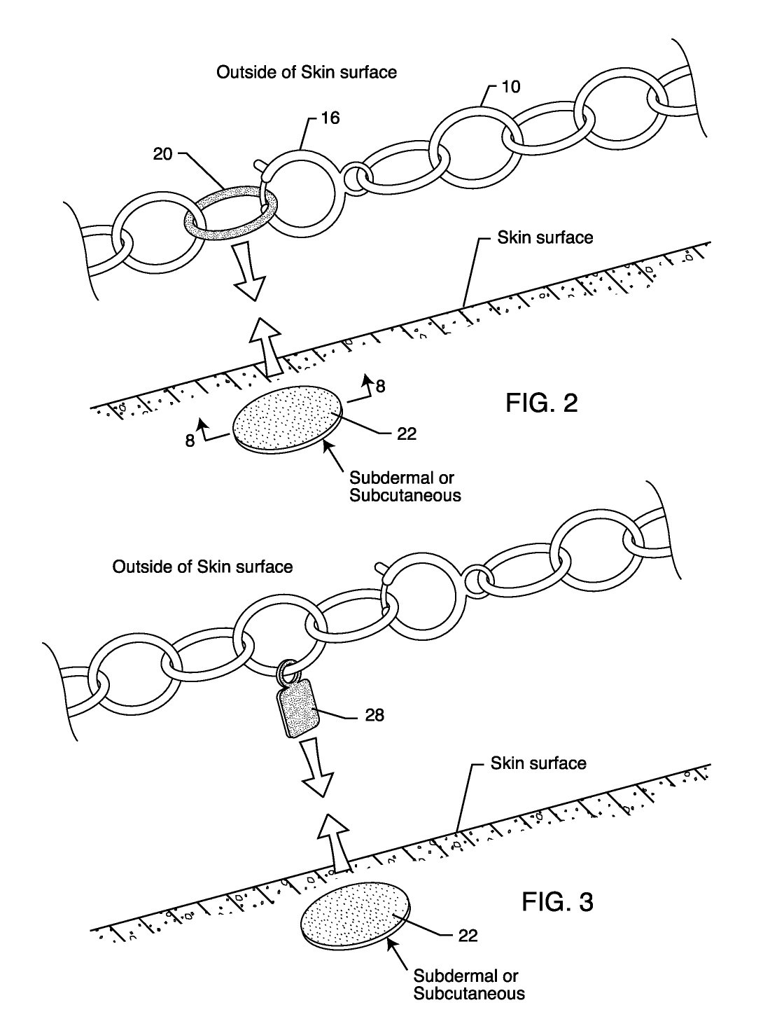

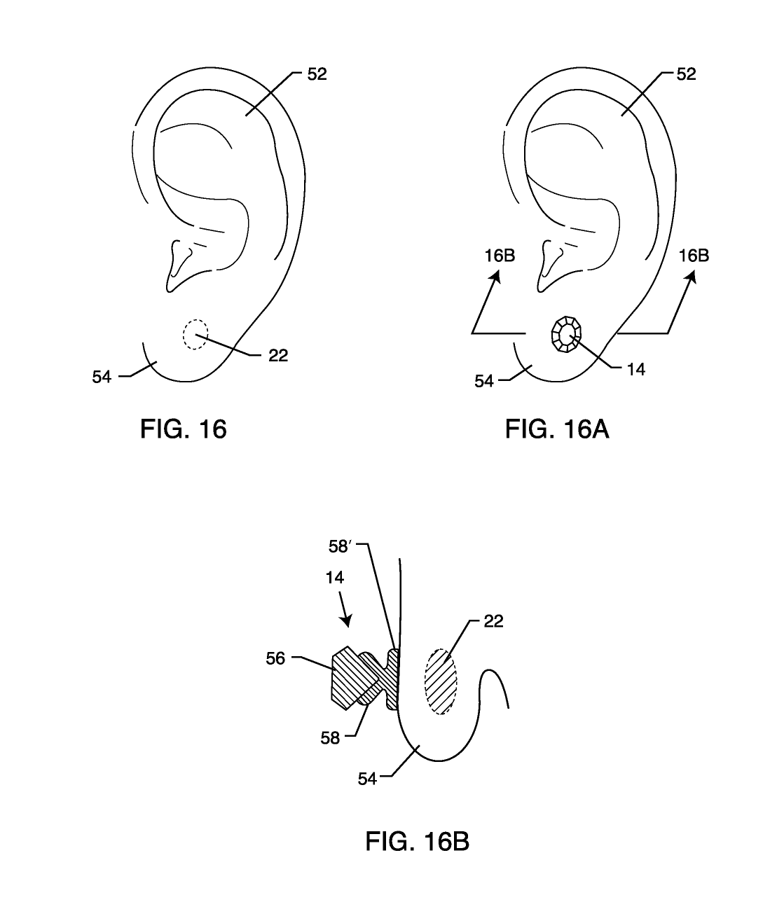

FIG. 2 illustrates a close-up view of a necklace 10 and a typical clasp 16 as previously illustrated in the prior art FIG. 1 now with the present invention. Referring once again to FIG. 2, one can see a novel ring 20 which would consist of either a magnetic or a ferromagnetic material that has been attached in line with the chain. It can have a clasp-like structure very similar to FIG. 16 or it could be pre-manufactured as part of the necklace 10. This ring 20 is designed to work in conjunction with a subdermal or subcutaneous magnetic or ferromagnetic material 22, which is located just below the skin's surface. In this case, this disk would be placed in the back of the woman's 12 neck. Through trying on the necklace and making a small mark (with a surgical permanent marker), one could identify the optimal location for the insertion of the sub-dermal or subcutaneous magnetic or ferromagnetic disk material 22. It is noted that subdermal and subcutaneous have essentially the same meaning as it means it is placed within the skin or under the skin.

Referring once again to the implanted disk 22 of the present invention, it will be either ferromagnetic, magnetic or a combination of the two. This will lead to some minor problems during medical diagnostic imaging, such as magnetic resonance imaging (MRI). The implantable structures 22 of the present invention are not long enough to effectively couple to the MRI RF resonant field. Accordingly, they will not present any kind of an overheating problem during the MRI procedure itself. However, some local image artifact can be expected in the area of the implant. The amount of image artifact is expected to be very low. It is encouraging that in recent years, MRI imaging sequences have been developed to minimize image artifact, for example, the artifact that's around an implantable medical device, such as a cardiac pacemaker. One also has to consider the powerful static magnetic field of an MRI machine. The most common MRI machines currently in use are 1.5 Tesla. There are also many 3 Tesla machines in the marketplace. This will definitely exert a pull-force on the implant 22. This is one of the main reasons why the implants have been provided with substantially large surface areas, and incorporate methods for tissue ingrowth. For example, it was a fear in the cardiac pacemaker industry that enough force would be exerted to actually have an implant be dislodged or even come out of the human body. These fears have proven to be false, and have also been greatly reduced with the ongoing miniaturization of active implantable medical devices. Also, the use of less magnetic components or ferromagnetic components in active implantable medical devices has helped.

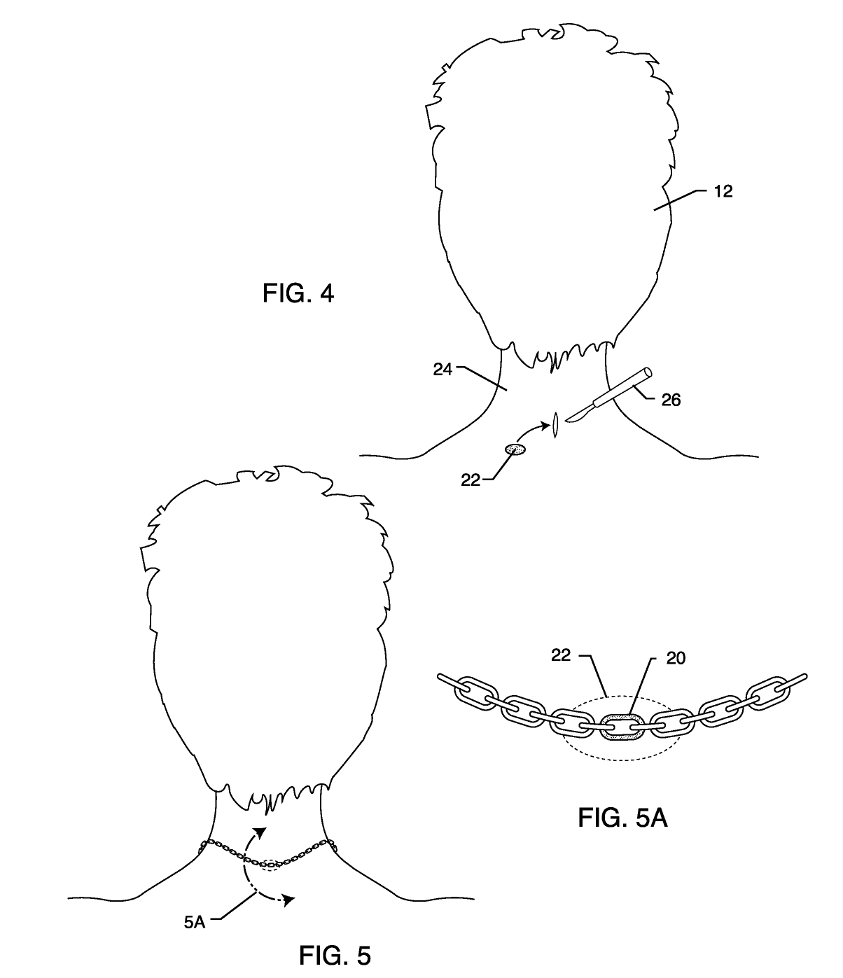

FIG. 4 shows the back of the woman's 12 neck 24 showing a scalpel 26 that has made a very thin incision which is not very deep. By “not very deep,” we mean just through the skin layer in which the magnetic or ferromagnetic disk 22 is then inserted. After insertion of the disk, typically the patient would wear a Band-Aid for about a week allowing the very small incision to heal. At this time, the patient is ready to wear their necklace, as shown in FIG. 5 wherein the clasp area of the necklace is magnetically held due to the magnetic attraction forces between the ring 20 and the sub-dermal or subcutaneous disk 22. It should be pointed out that the invention is reversible. That is, the magnet may be inserted subcutaneously or subdermally or the magnet may be affixed to the necklace itself or two magnets could be used where there are oriented such that they have a north and south polarity, which would give them optimal attraction. Ideally, one wants about 0.5 to 4.5 lbs. of pull force between the necklace 20 and the inserted disk 22.

FIG. 5 shows that as a patient moves throughout the day, the clasp area will remain held tightly to the sub-dermal or subcutaneous disk implanted in the skin at the back of the neck.

FIG. 5A is a blown up taken from FIG. 5 and shows that the necklace clasp area is being held very firmly in place over the implanted disk 22.

FIG. 3 shows an alternative form of a ferromagnetic attachment piece 28 which lays flat against the implanted disk 22. Referring once again to FIG. 1, it is typical that the necklace be of precious metal, such as gold or platinum. This is important not only for beauty, but also such that woman 12 can enjoy wearing her necklace for long periods of time without fears about bio-compatibility, her neck turning green or the like. It is therefore very important that any material that is added to the necklace, such as ring 20 shown in FIG. 2 or flat plate 28 shown in FIG. 3, also be of long-term non-toxic and biostable construction. Fortunately, U.S. Navy and MIL SPECS have addressed this issue. For example, if ring 20 is ferromagnetic containing iron, it would be plated first with a layer of copper and then a layer of nickel as a barrier layer and then plated with ultrapure or jewelry grade gold of a sufficient thickness of >50 millionths of an inch so that over the life of the necklace, it never becomes toxic or appears different in appearance than the rest of the necklace 10. Of course, the same thing is true for the flat plate magnet or ferromagnetic material 28 shown in FIG. 3.

Referring once again to FIGS. 2 and 3, it is also very important that the implanted disk be completely long-term biocompatible, non-toxic and biostable. It is also very important that it be physically strong. Magnetic materials tend to be very brittle and they are easy to crack. Accordingly, the magnetic material 22 as illustrated in FIGS. 2 and 3 must have sufficient structural strength so that it not be inadvertently damaged. It will be shown in subsequent drawings how this material can be made to be biocompatible, biostable and non-toxic while at the same time providing structural rigidity.

It is also important that the inserted disk not migrate over time inside the patient's body. For example, in the pacemaker industry, it has been shown that people often fiddle or twiddle with their implanted device. There are actual technical papers published called, TWIDDLER'S SYNDROME regarding pacemaker implants where over time, the patient has managed to spin it around in a clockwise or counter clockwise direction as much as 5 or 6 times, even up to the point of breaking a lead. This analogy is appropriate to the present invention wherein it is very important that the disk be structurally strong and also be made in a way in which it will not migrate.

FIG. 6 illustrates a modification of the implanted disk 22′ wherein a number of through holes 30 have been provided for tissue ingrowth. During healing, this allows scar tissue and other tissues to form between the top and the bottoms of the disk thereby making it either impervious or less sensitive to migration after implant healing.

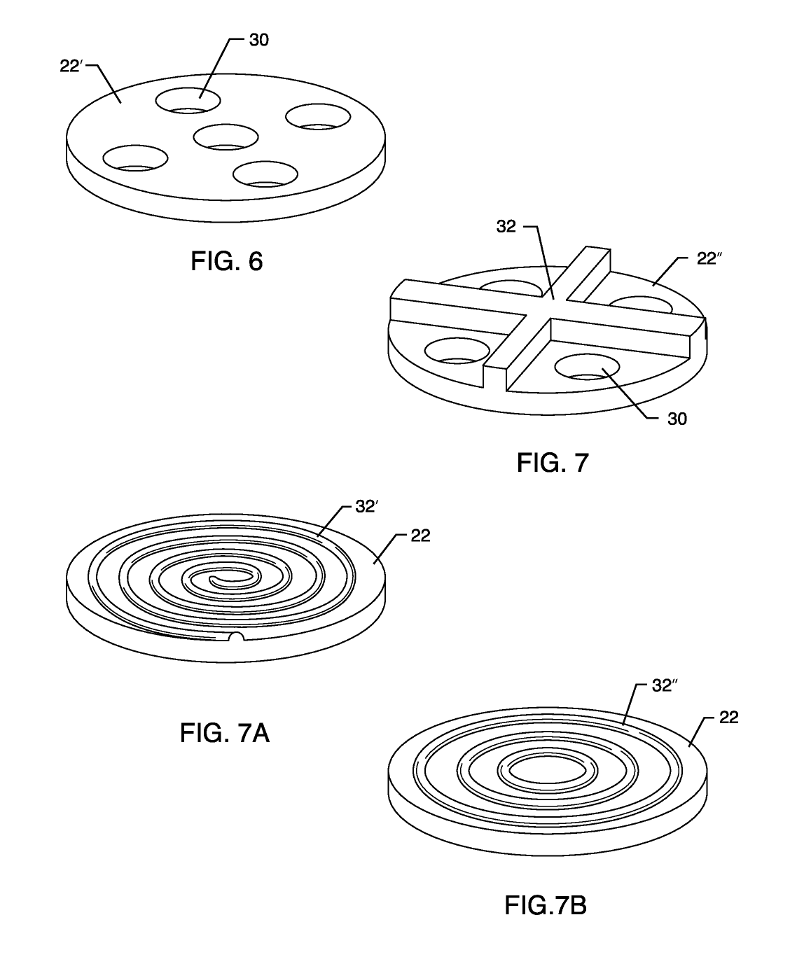

FIG. 7 is very similar to the disk 22″ previously illustrated in FIGS. 2, 3 and 6 except that in addition to tissue ingrowth holes 30, stiffening ribs 32 have been added to provide more structural rigidity to make the entire implanted disk less sensitive to breakage and fracturing during an impact or handling. It will be understood to those skilled in the art that these ribs 32 can take on a number of alternative shapes, including circular grooves, circular bumps or other forms of ridges. Shown in FIGS. 7A and 7B, spiral or circular ridges would also add structural rigidity while keeping the mass of the implant 22 low.

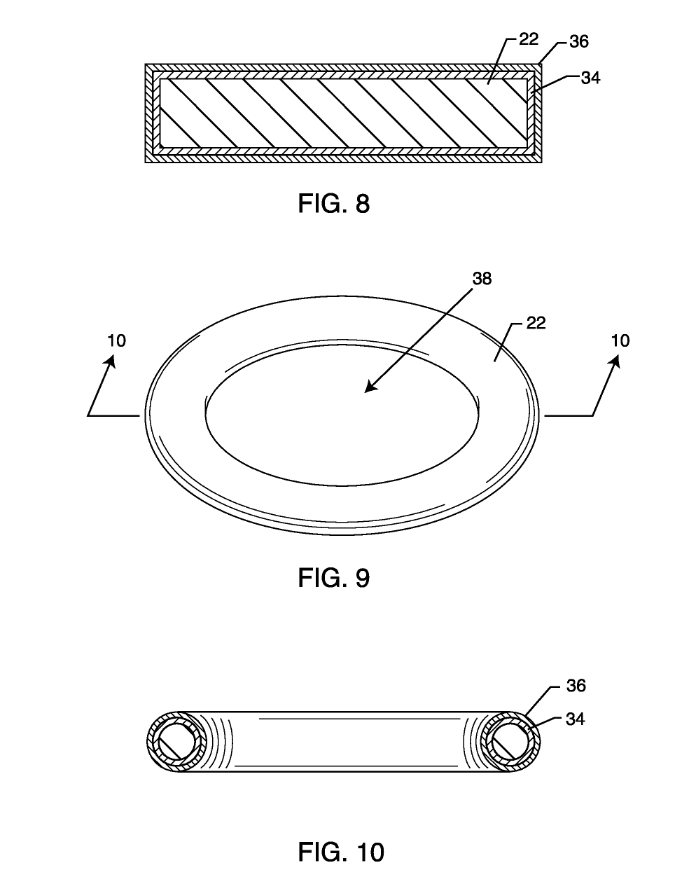

FIG. 8 is taken generally from section 8-8 from FIG. 2 and shows the disk 22 in cross-section. In this case, there is a biomedical sputter or deposited coating 34, such as alumina ceramic that is placed on top of the metallic or ferromagnetic or magnetic disk 22. There is a second layer of protection 36, such as plasma etched vapor deposited paralyne. What is really important is that the disk be coated with suitable and durable biocompatible materials that will remain biostable over the full life of the implant, which of course, could be as much as 100 years or more.

The protective material can comprise titanium or other metal material plated, deposited, or otherwise coated upon the magnetic material. As another example, the protective material can include a parylene coating, silicone polymer, a non-toxic epoxy, a medical grade polyurethane, or a U.V. curable medical acrylic copolymer. The protective coating may also incorporate anticoagulants and/or antibiotics and/or tissue in-growth promoters.

There have been a few attempts at implantable magnets yet they completely underestimated the challenges of long-term biocompatibility in a hostile environment of the human body. These attempts have included using a neodymium-iron boron alloy with a thin gold plating encapsulated in silicone. For a while, people who received these implants were satisfied. However, the main problems have been caused by the rupturing of the silicone covering bringing metal into contact with body tissues which in most cases cause it to rapidly break down. Other attempts were made using injected molded silicone rather than dipped coating. This led to thin spots where the silicone was more likely to break down and cause failure. As such, these second generations were another naïve attempt at a magnetic implant.

In order for the present invention to be successful, one must carefully consider the following: long-term biocompatibility; non-toxicity; biostability; mechanical strength; allergies and the possibility of implant rejection; and migration or dislodgement. The present invention addresses every one of these needs through the use of well-known stable materials that have a proven track record of biocompatibility. In the example previously described in FIG. 8, sputtering of alumina followed by a layer of polyimide makes use of two well documented layers which would provide long-term biocompatibility and non-toxicity.

FIG. 9 illustrates the implanted ring 22 previously illustrated in FIG. 2 except that this time it is round or donut shaped. The large aperture or hole through the center 38 allows for maximum tissue ingrowth.

FIG. 10 is a sectional view 10-10 taken from FIG. 9 and shows that there are biocompatible layers 34 and 36 as previously described in FIG. 8.

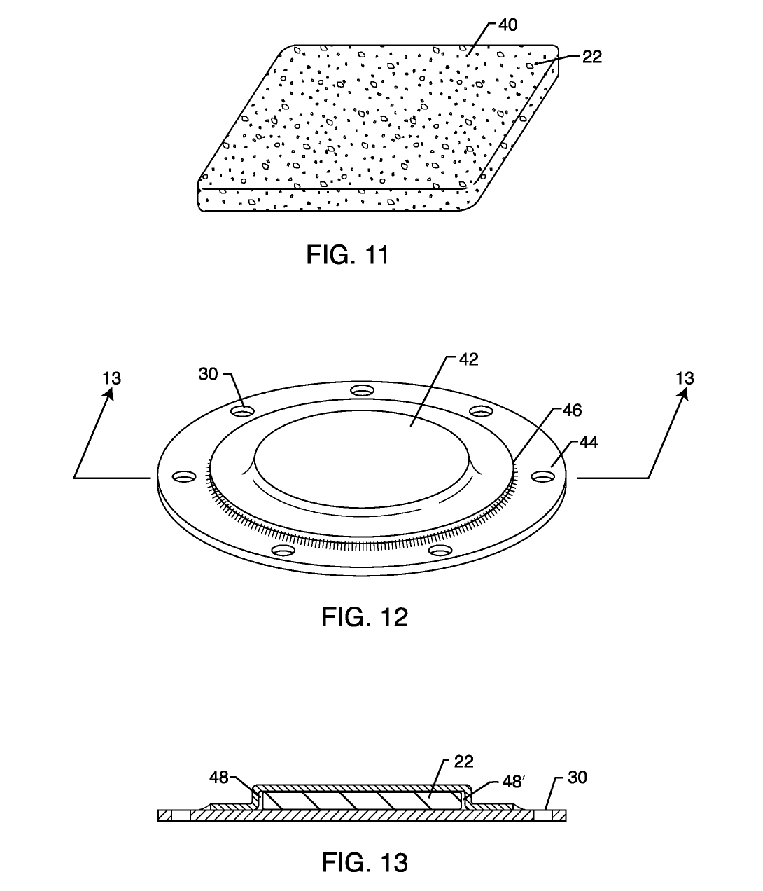

FIG. 11 illustrates that the implantable disk 22 can be made of a microporous material that literally has hundreds, if not thousands, of holes for tissue ingrowth. FIG. 11 also illustrates that various geometries can be used (in this case, square). It will be understood that one can use any geometry, such as oval, round, elliptical, and the like for the shape of the implanted disk. It would be undesirable to have sharp corners in the implanted disk. For example, it would not be a preferred embodiment to have it triangular in shape with sharp points. Sharp points would lead to pain, migration and other undesirable effects as the skin flexes.

FIG. 12 illustrates another embodiment of the present invention. In FIG. 12, there is a top stamped disk out of titanium, platinum, gold, ceramic or other biocompatible metal or material 42. This is laser welded 46 to a lower disk 44 out of similar materials. The laser weld 46 is done in an inert gas and forms a hermetic seal. This is best understood by referring to the cross-sectional view shown in FIG. 13, which is taken generally from section 13-13 from FIG. 12. Shown in cross-section is the implantable pro-magnetic or magnetic disk 22 which is now completely enclosed by structurally strong and known to be biocompatible materials. A number of tissue ingrowth holes 30 are provided to prevent migration of the device subcutaneously. For cosmetic purposes, it is very important that the assembly be kept very thin. This is to avoid an unsightly bulge under the skin. For this purpose, as is used in the pacemaker industry, titanium is an ideal choice of materials. Not only does it have proven long-term biocompatibility, but it is also, for its thickness and weight, one of the strongest materials known. Referring back to FIG. 12, the top plate 42 and bottom plate 44 could also be of alumina ceramic or other ceramic materials. In another embodiment, at least 96% pure alumina ceramic would be used and the co-joining 46 would again be done by a sputtered surface and a laser weld or by firing a glass seal in elevated temperature between the two surfaces to form a hermetic seal. Another advantage to the structure as illustrated in FIGS. 12 and 13 is that, for example, powerful neodymium magnet 22 could be used, which would be relatively small in diameter and thickness. It would be mechanically supported by the much larger area of the bottom plate 44, which would distribute structural loads and stresses over a much larger area. For example, if one were to hang an earring type structure or necklace type structure on this arrangement, the forces subcutaneously would be spread over a large area and therefore the implant would be held very firmly in place. The bottom plate can be as much as 2×, 3×, 4× or even 10× the largest dimension of the surface area of the magnetic or ferromagnetic implant 22. Referring once again to FIG. 12, the laser joint 46 could be replaced by a precious metal braze or weld, such as a gold braze or the like.

In the drawing description for FIG. 12, it was mentioned that the laser weld 46 would be ideally done in a chamber back-filled with nitrogen. It would be desirable to also put in a helium tracer gas in the nitrogen; so that after sealing, one could readily do an automated hermetic seal test. In other words, after cleaning the entire structure could be sniffed by a helium leak detector for any trace of helium, which would indicate pin holes or voids in the continuous laser weld 46. The process is better described as follows: the subassembly as shown in FIG. 12 (prior to laser welding 46), would be placed into a robotically controlled welding chamber. A vacuum would be pulled thereby pulling the air out of any interior spaces 48 and 48′, such as those around the magnetic implant 22 shown in the sectional view of FIG. 13. The pulling of vacuum is generally done in an elevated temperature to make sure that all of the air has been removed from the assembly. Then the chamber is back-filled with dry nitrogen with a helium tracer gas. At this time, the laser performs laser weld 46 hermetically sealing the assembly, which now has nitrogen with the tracer gas permanently trapped within it. An alternative to this would be an argon welding process.

Referring once again to FIG. 13, one can see that the addition of the top plate 42 and the bottom plate 44 does add substantial thickness to the overall implanted disk 22. However, in the hands of a skilled surgeon or even a tattoo artist, this is relatively easy to accommodate. For example, if this was being inserted in the back of the neck, a tiny incision would be made, a small amount of local fat would be removed and then the disk 22 inserted into position. For other body locations, for example, on the side of the nose. Just before the lateral nasal cartilages, the greater Alar cartilages is present, which is a thin flexible plate that forms the medial and lateral wall of the nostril. In addition to the Alar cartilages, there are three or four small cartilages that are called lesser Alar cartilages. Both the greater and the lesser Alar cartilage give the over shape of the nostrils. Surrounding these areas are soft tissues, such as the entra-tip and lobulous, which are ideal areas to insert the disk 22 of the present invention. It is also easy to remove a little bit of cartilage or tissue from these areas thereby making room for the thickness of the insert 22 so that after healing, there is no bump or negative cosmetic consequences to the insert.

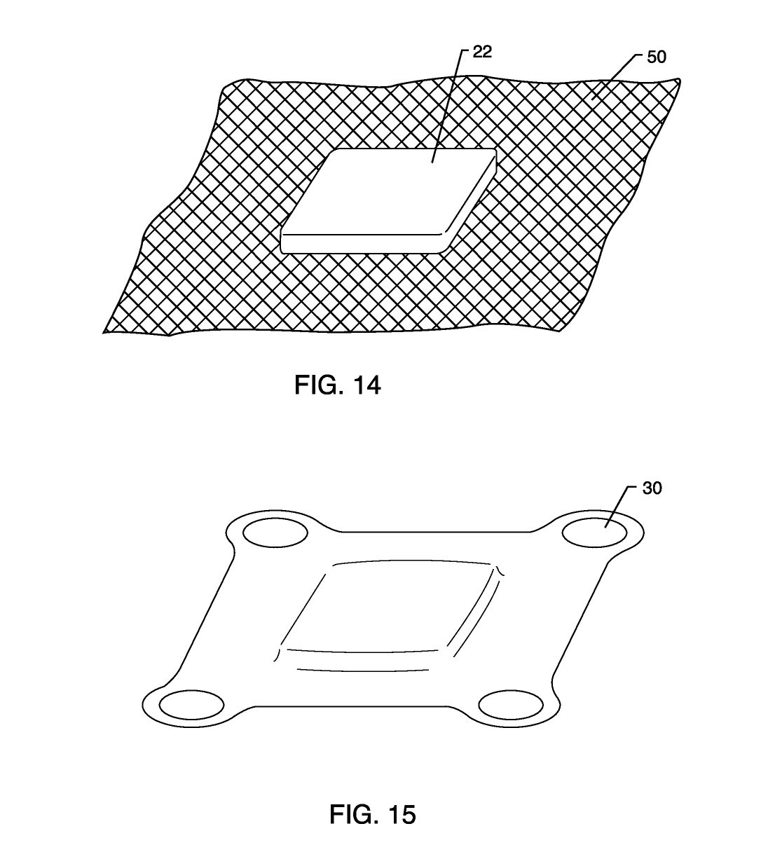

FIG. 14 shows the implantable disk 22 of the present invention, which has been co-bonded to a matrix substrate 50. The long-term biocompatible adhesive is used (not shown) to affix the implantable magnet or ferromagnetic material 22. The disk 50 is in the form of a GORE-TEX or Medpore or other biocompatible mesh, which facilitates a great deal of tissue ingrowth. This not only prevents the disk 22 from migrating, but also provides a lot of overall structural strength to the entire area.

FIG. 15 is an alternative view taken from FIG. 14 showing the addition of four holes 30 to further facilitate tissue ingrowth.

FIG. 16 is a side view of a human ear 52 showing an earlobe 54. Shown is the insert 22 of the present invention.

FIG. 16A shows the addition of an ornament or jewelry, such as a diamond 14.

FIG. 16B is taken from section 16B-16B from FIG. 16A showing the implanted disk 22 and a side view of the jewelry, which in this case contains a diamond 56 which is held in a setting 58, which has a base 58′ which is either ferromagnetic, magnetic or the like in accordance with the present invention. An advantage of the structure as illustrated in FIG. 16B and the other figures herein, is that a very substantial pull-force is exerted between the base plate 58′ and the implantable disk 22. As previously mentioned, it is desirable that this pull strength be at least 0.5 to 4.5 lbs. A half pound to four and a half pounds is sufficient pull-force where when one goes to remove the diamond, one will literally end up pulling their nose or their earlobe way to the side before the jewelry ornament 14 breaks loose. However, 0.5 to 4.5 lbs. of pull-force is not so much that the implant would cause discomfort or be dislodged or ripped out of the ear. This is a major downside in the prior art wherein a piercing with a backing plate is used. It has been well documented that many serious stud injuries have occurred both with nasal piercings, ear piercings, belly button piercings, nipple piercings and the like. Just a simple tussle or playful rough-housing can cause a sweater to pull against one of these implants and literally rip it from the skin leaving behind a large rip in need of surgical and cosmetic surgeon repair.

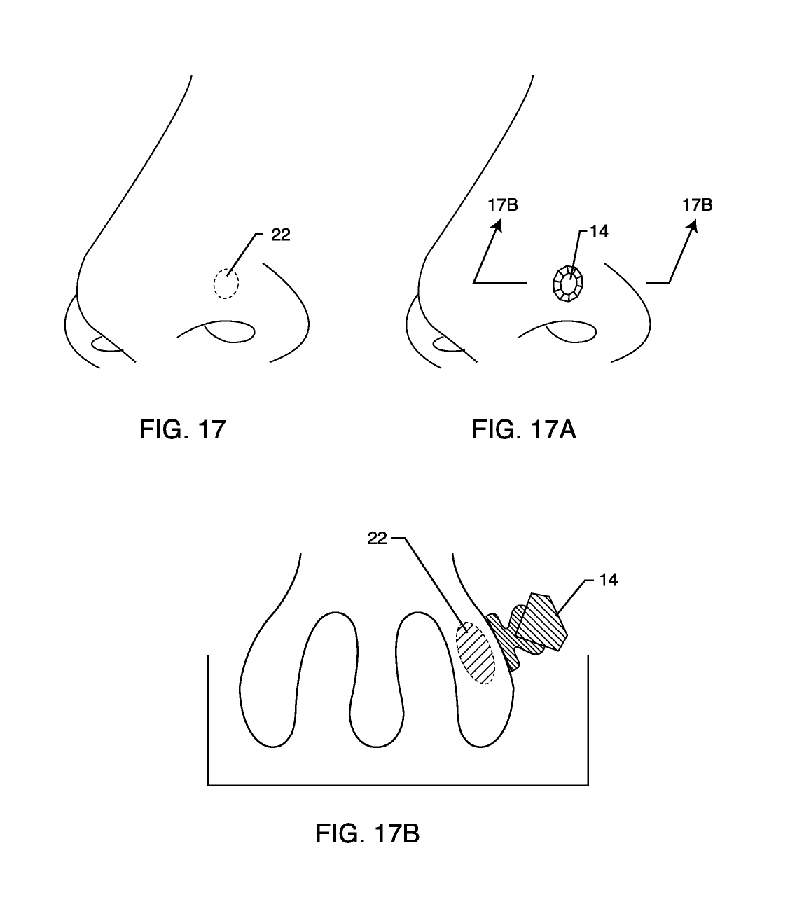

FIG. 17 is the front view of a human nose 68 with an implant 22 of the present invention as shown subcutaneously. Also shown, is an item of jewelry 14 similar to that already described in FIG. 16B.

FIG. 17B is taken generally from section 17B-17B from FIG. 17A which shows the item of jewelry held firmly in place magnetically to the implantable disk 22. Another advantage to these features of the present invention is it becomes very easy to switch items of jewelry 44 or not even wear them at all. That is not an option for somebody with a nose piercing now. Removal of the item of jewelry leaves an unsightly hole left behind that must be covered. Referring once again to FIGS. 17, 17A and 17B, there is another major downside to having a permanent healed hole in the side of your nose. Consider what happens when one has got a head cold with a runny discharge and goes to blow their nose and having fluid material blowing out the side.

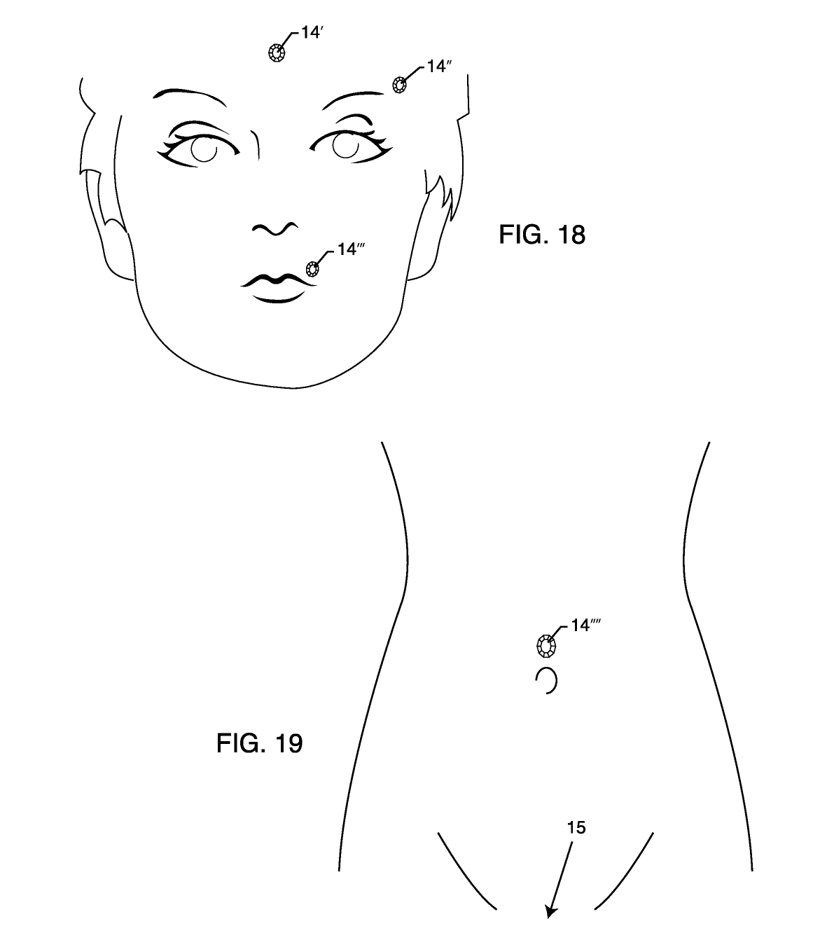

FIG. 18 shows alternative locations for the insert 22 and the mating item of jewelry 14′ and 14″ and 14′″ of the present invention. Jewelry item 14′, for example, could be just a simple red dot, which is common in Indian culture or it could be a red ruby or the like. Jewelry item 14″ replaces a very commonly used ring known as an eyebrow ring. Jewelry item 14′″ replaces a common type of lip piercing, which can be on the upper lip as shown or on the lower lip (not shown). Also not shown, is an insert into the tongue so that one may wear the item of jewelry 14 against the tip or any other location of the tongue. It should be noted that the present inventor does not consider an implant in the tongue to be particularly ideal since if the item of jewelry 14 became dislodged, it could easily be swallowed. In addition, the inside of the mouth is an area that is very active in bacteria making any type of piercing or implant in that area problematic.

FIG. 19 illustrates a navel implant 14″″. It will be obvious that in FIG. 19, the implanted disk 22 can be placed through a small incision made inside the belly button so that it becomes completely invisible. If one imagines a clock face from zero to 12:00, the implant 22 can be placed anywhere around the belly button and around that clock face. For reasons of delicacy, it will be understood that the inserted disk could be placed anywhere on the genitalia of either the male or female body 15 as can be generally imagined. Furthermore, it will be understood that the present invention can be placed at a variety of locations within the human body not specifically covered herein, such as the toe, the foot, the top of foot, the ankle, the calf, the knee, the thigh, the arm, the torso, the neck, etc.

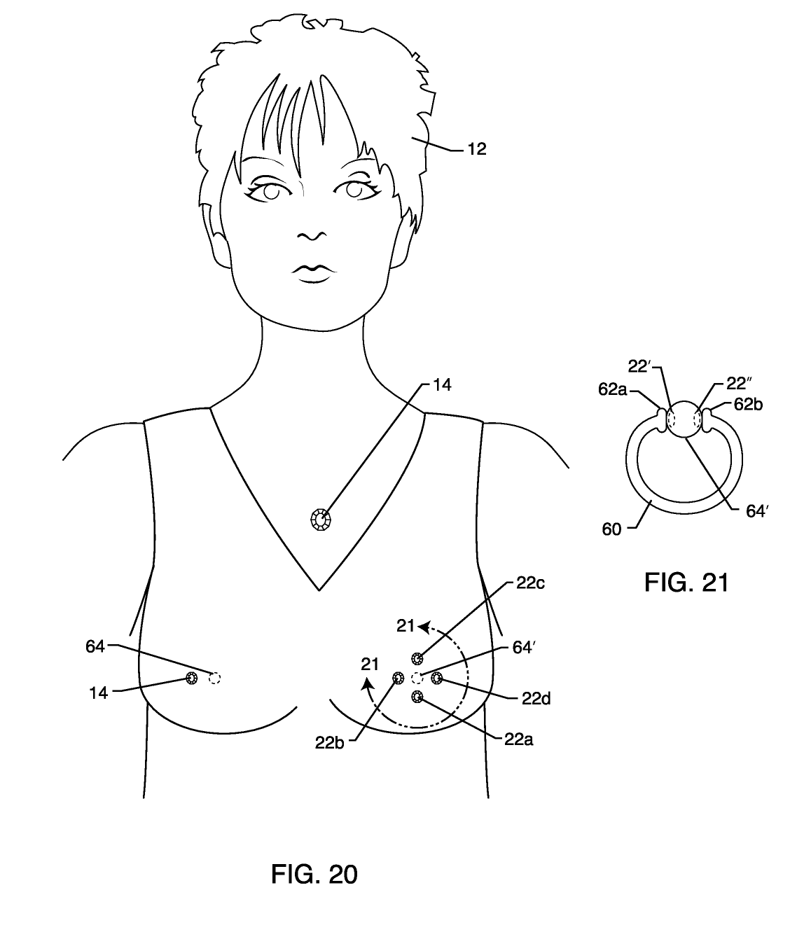

FIG. 20 illustrates that the insertable disk 22 of the present invention can be placed at or adjacent or even into either or both nipples of a male or a female human 12. As previously described for the belly button, if one imagines a clock face, the insert around the nipple of FIG. 20 can be in any position of the clock around the nipple 64. Referring once again to FIG. 20, one can see that the subcutaneous insert can be “clocked” in any position around the nipple 64′ as shown as 22a, 22b and 22c.

FIG. 21 is taken generally from section 21-21 from FIG. 20 showing a close-up view of a human nipple 64′. In this case, there are two subcutaneous inserts 22′ and 22″. These are designed to electromagnetically couple with area 62a and 62b, which are small flattened sections on each end of the ring structure 60. The idea here is to simulate a ring nipple piercing without the actual piercing. Locations for the implant 22 of the present invention are limited only by one's imagination. They not apply to every type of human, but also to any other type of animal or pet that could be imagined.

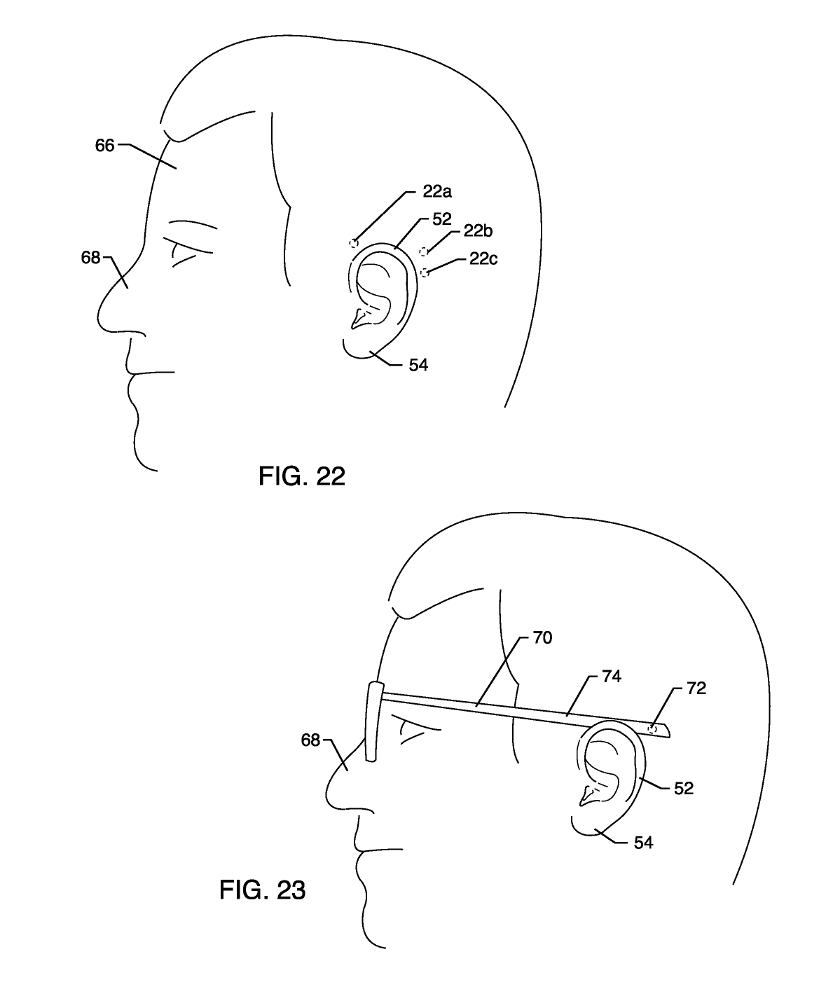

FIGS. 22 and 23 are side views of a person's head 66 with nose 68. It is very common for eyeglasses 70 to repeatedly slip down the bridge of one's nose. This is especially true during sporting activities or when the head is in motion. This can become quite frustrating having to repeatedly push and reset one's eyeglasses 70. For this reason, prior art eyeglasses are usually bent way around behind the ear 52, which can be very uncomfortable and hard to get used to.

FIG. 22 is a side view of a male human head 66 that is being prepared for a special pair of eyeglasses. The eyeglasses 70 may have a disk 72 that is integrated into the temple 74 of the eyeglasses 70. This disk 72 can take the shape of a circle, as shown, or a long rectangle (not shown). Implantable magnetic or ferromagnetic disk 22a, 22b and 22c are shown “clocked” around the ear 52 in FIG. 22. Eyeglasses generally consist of a bridge piece, which is placed directly over the nose and then two hinges and two temple pieces. Temple piece 74 is indicated in FIG. 23. The disk 72 is magnetically attracted to the disks 22 of the present invention as taught herein. In this manner, a force is created that helps to hold the eyeglasses 70 in place.

It will be understood to those skilled in the art that the disk 72 may be movably adjusted along the temple 74 of the eyeglass 70. The disk can be placed or moved between various receptacles or the disk may be translated along the temple 74 with a screw-type arrangement. Fine tuning of the placement of the disk 72 within the temple 74 of the eyeglasses 70 allows the wearer to fine tune the amount of force being exerted.

In the present invention, any type of temple shape, including straight as shown 74, is appropriate. This is because the eyeglasses will be held firmly in place by the magnetic attraction between the disk 72 and the implant 22. In an alternative location for the ferromagnetic or magnetic insert would be right over the bridge of the eyeglasses (not shown). The bridge sits right on top of the nose. An insert 22, in accordance with the present invention, could be inserted right over the bridge area of the nose, such that the eyeglasses were also held firmly in place in that location. This is definitely not the preferred embodiment for cosmetic reasons. It is well known in the cochlear implant art that it is very easy to create a skin flap behind the ears and insert a metallic object. Accordingly, another location for the insert 22 would be behind the right and left ear locations as illustrated in FIG. 22.

Although several embodiments have been described in detail for purposes of illustration, various modifications may be made to each without departing from the scope and spirit of the invention. Accordingly, the invention is not to be limited, except as by the appended claims.

What is claimed is:

1. An implantable magnetic retention system, comprising:
an implantable portion configured to be implanted subdermally or subcutaneously within a patient, the implantable portion comprising a magnetic or ferromagnetic inner portion and a biocompatible outer portion fully enclosing the inner portion; and
a non-implantable portion configured to be attached to a wearable ornament, the non-implantable portion comprising a magnetic or ferromagnetic portion;
wherein at least one of either the implantable portion or the non-implantable portion comprises the magnetic portion; and
wherein the implantable portion and the non-implantable portion are magnetically attracted to one another.

2. The system of claim 1, wherein the implantable portion is separate and distinct from the non-implantable portion.

3. The system of claim 1, wherein the implantable portion and the non-implantable portion comprise between 0.5 to 4.5 lbs of pull force when abuttingly disposed to one another.

4. The system of claim 1, wherein the wearable ornament comprises a necklace, a finger ring, a toe ring, an eyebrow ring, a belly ring, a nipple ring, a precious gemstone, an ornamental figure, a trinket or a pair of eyeglasses.

5. The system of claim 1, wherein the biocompatible outer portion is biocompatible, biostable and non-toxic.

6. The system of claim 1, wherein the biocompatible outer portion comprises a first layer of copper, where the copper is fully enclosed by a second layer of nickel, where the nickel fully enclosed by a third layer of gold.

7. The system of claim 6, wherein the gold layer is at least 50 millionths of an inch thick throughout.

8. The system of claim 1, wherein the implantable portion comprises at least one hole disposed therethrough.

9. The system of claim 1, wherein the implantable portion comprises a plurality of holes disposed therethrough.

10. The system of claim 1, wherein the implantable portion comprises at least one rib formed therein, wherein the rib is straight, curved, circular or spiral shaped.

11. The system of claim 1, wherein the implantable portion is ring shaped.

12. The system of claim 1, wherein the implantable portion includes a microporous material.

13. The system of claim 1, wherein the implantable portion's biocompatible outer portion comprises a top portion sealed to a bottom portion by a seal.

14. The system of claim 13, wherein the seal comprises a laser weld, a glass seal or a precious metal braze.

15. The system of claim 13, wherein the top portion and bottom portion both comprise titanium, gold or ceramic.

16. The system of claim 13, wherein at least one of the top or bottoms portions have a surface area two times the surface area of the inner portion.

17. The system of claim 13, wherein at least one of the top or bottoms portions have a surface area four times the surface area of the inner portion.

18. The system of claim 13, wherein at least one of the top or bottoms portions have a surface area ten times the surface area of the inner portion.

19. The system of claim 1, wherein the implantable portion further comprises a biocompatible mesh substrate attached to the outer portion.

20. The system of claim 1, wherein the biocompatible outer portion comprises a first layer fully enclosed by a second layer, where the first layer comprises a biomedical sputter or deposited coating.

21. The system of claim 20, wherein the first layer comprises alumina ceramic.

22. The system of claim 20, wherein the second layer comprises plasma etched vapor deposite paralyne, titanium, silicone polymer, non-toxic epoxy, medical grade polyurethane, or U.V. curable medical acrylic copolymer.

23. The system of claim 1, wherein biocompatible outer portion comprises an anticoagulant, an antibiotic or a tissue in-growth promoters.

24. An implantable magnetic retention system, comprising:
an implantable portion configured to be implanted subdermally or subcutaneously within a patient, the implantable portion comprising a magnetic inner portion and a biocompatible outer portion fully enclosing the inner portion, where the biocompatible outer portion comprises a first layer fully enclosing the magnetic inner portion and a second layer fully enclosing the first layer; and
a non-implantable portion attached to a wearable ornament, the non-implantable portion comprising a magnetic or ferromagnetic portion;
wherein the implantable portion and the non-implantable portion are magnetically attracted to one another.

25. An implantable magnetic retention system, comprising:
an implantable portion configured to be implanted subdermally or subcutaneously within a patient, the implantable portion comprising a magnetic or ferromagnetic inner portion and a biocompatible outer portion fully enclosing the inner portion, where the biocompatible outer portion comprises a first layer fully enclosing the magnetic inner portion and a second layer fully enclosing the first layer; and
a non-implantable portion configured to be attached to a wearable ornament, the non-implantable portion comprising a magnetic portion;
wherein the implantable portion and the non-implantable portion are magnetically attracted to one another.
